(19) United States
(12) Patent Application Publication
(10) Pub. No.: US 2017/0231768 A1
(43) Pub. Date: Aug. 17, 2017

(54) MAGNETIC JOINT TREATMENT DEVICE

(71) Applicant: RAINBOW MEDICAL LTD., Herzliya (IL)

(72) Inventor: Yossi GROSS, Moshav Mazor (IL)

(73) Assignee: RAINBOW MEDICAL LTD., Herzliya (IL)

(21) Appl. No.: 15/042,841

(22) Filed: Feb. 12, 2016

(51) Int. Cl. A61F 2/30

(52) U.S. Cl. CPC A61F 2/30; A61F 2002/30079; A61F 2002/30673

(57) ABSTRACT

Apparatus is provided for treating a native joint of a subject. The apparatus comprises a flexible cuff shaped and sized to be worn, at least in part, around the native joint of the subject. A magnet is coupled to the flexible cuff such that when the flexible cuff is worn by the subject, the magnet is positioned within 8 cm, e.g., within 6 cm, from a joint cavity of the native joint. The apparatus additionally comprises a ferrofluid for placing in the joint cavity. The ferrofluid is typically maintained in place within the joint cavity by the magnet which is coupled to the cuff. Other applications are also described.

### FIELD OF THE INVENTION

Embodiments of the present invention relate generally to medical devices and more particularly to methods and apparatus for treatment of a native joint of a subject.

### BACKGROUND

Synovial joints, e.g., knees, elbows, shoulders and hips, are typically comprised of two or more bones that meet at a common point, but remain structurally separated from each other, in order to allow motion of body parts. Typically, the ends of the articulated bones, i.e., the opposing surfaces where the bones meet, each have a surface of cartilage that limits friction between the bones and allows the bones to glide smoothly against each other. Additionally surrounding the ends of the two articulated bones is synovial fluid that functions as an additional lubricant to reduce friction between the ends of the bones that rub together in the joint. The synovial fluid is typically a viscous, hyaluronic acid-containing fluid, that is found in cavities of synovial joints and is contained within a joint capsule.

Osteoarthritis (OA), also known as degenerative joint disease, is a type of joint disease that results from erosion of joint cartilage and underlying bone, and thinning of the synovial fluid. Osteoarthritis typically causes joint pain, stiffness and decreased range of motion. Hyaluronic acid joint injections are a common approach for providing short term pain relief from osteoarthritis. Alternately, joint replacement surgery offers a long-term treatment option by replacing the defective joint with an artificial joint.

A ferrofluid is a liquid that becomes strongly magnetized in the presence of a magnetic field.

### SUMMARY OF THE INVENTION

In accordance with some applications of the present invention, a method is provided for treatment of a native joint of a subject. In particular, some applications of the present invention provide a method and apparatus for holding in place fluid that is injected into a native joint cavity of a subject.

Typically, a magnet is implanted within a bone of the subject, e.g., at a site that is within 3 cm from the joint cavity, and a ferrofluid is placed, e.g., by injection, in the joint cavity of the native joint. The ferrofluid is magnetized by the magnet, and is thereby kept in place within the joint cavity, near the magnet. The ferrofluid is typically kept in place by the magnet such that leakage of the ferrofluid from the joint cavity is inhibited. Additionally or alternatively, at least a portion of the ferrofluid is maintained in proximity to the magnet such that the ferrofluid is kept in a desired location within the joint cavity.

The ferrofluid is typically placed in the joint cavity in order to function as a lubricant in cases in which there is thinning of the native synovial fluid and/or degeneration of joint cartilage. In other words, the ferrofluid acts as a bearing which reduces friction between articulating bones in the native joint in cases of degenerative joint diseases.

Injecting ferrofluid in the joint cavity and maintaining the ferrofluid within the joint cavity by placement of a magnet in an adjacent bone typically provides a longer-lasting and/or more effective joint injection treatment compared to commonly-used joint injection treatments for treating degenerative joint diseases. Typically, prior art joint injections provide only relatively-short pain relief, perhaps due to slow leakage of the injected fluid from the joint cavity. In contrast, as provided by some applications of the present invention, placing a ferrofluid in the joint cavity and inhibiting displacement of the ferrofluid by using an implanted magnet, offers a longer-lasting joint injection and may thereby postpone the need for additional injections.

Additionally or alternatively, placing a ferrofluid in the joint cavity and inhibiting displacement of the ferrofluid within the cavity by using an implanted magnet, in accordance with some applications of the present invention, delays or replaces the need for joint replacement surgery.

There is therefore provided in accordance with some applications of the present invention, apparatus, including:

a flexible cuff shaped and sized to be worn, at least in part, around a native joint of a subject;

a magnet coupled to the flexible cuff such that when the flexible cuff is worn by the subject, the magnet is positioned within 8 cm from a joint cavity of the native joint; and

a ferrofluid for placing in the joint cavity.

For some applications, the magnet includes a flexible magnet.

For some applications, the magnet includes a rigid magnet.

For some applications, the magnet is configured to be positioned within 6 cm from a joint cavity of the native joint.

There is further provided in accordance with some applications of the present invention a method for treating a native joint of a subject, the method including:

placing a flexible cuff that includes a magnet around a native joint of a subject such that the magnet is positioned within 8 cm from a joint cavity of the native joint; and

placing a ferrofluid in the joint cavity of the native joint.

For some applications, placing the ferrofluid in the joint cavity includes placing 1-10 cc of the ferrofluid in the joint cavity.

For some applications, placing the flexible cuff includes placing the flexible cuff such that the magnet is positioned within 6 cm from the joint cavity.

There is further provided in accordance with some applications of the present invention a method for treating a native joint of a subject, including:

implanting a magnet within a bone of the subject that is adjacent to the native joint, at a site that is within 3 cm from a joint cavity of the native joint; and

placing a ferrofluid in the joint cavity of the native joint.

For some applications, placing the ferrofluid in the joint cavity does not include placing the ferrofluid in fluid communication with the magnet.

For some applications, the native joint is a native knee joint of the subject, and placing the ferrofluid in the joint cavity of the native joint includes placing the ferrofluid in a joint cavity of the native knee joint.

For some applications, implanting the magnet includes screwing the magnet into the bone.

For some applications, implanting the magnet includes implanting a diametrically-magnetized magnet.

For some applications, placing the ferrofluid in the joint cavity includes placing 1-10 cc of the ferrofluid in the joint cavity.

For some applications, implanting the magnet within the bone includes implanting the magnet 0.1-1 cm from the joint cavity.

For some applications, the magnet is a first magnet, and implanting the magnet within the bone includes implanting the first magnet, and the method further includes implanting a second magnet within a bone of the subject that is adjacent to the native joint, at a site that is within 3 cm from the joint cavity.

For some applications, implanting the first magnet and implanting the second magnet includes implanting the first and second magnets in the same bone of the subject.

For some applications, implanting the first magnet and implanting the second magnet includes implanting the first and second magnets in separate bones of the subject.

For some applications, implanting the magnet includes implanting a magnet having a diameter that is between 2 and 10 mm.

There is further provided in accordance with some applications of the present invention, apparatus including a diametrically-magnetized bone screw.

For some applications, the apparatus further includes a kit in which the bone screw is disposed, the kit further including a syringe loaded with a ferrofluid.

For some applications, the bone screw has a diameter that is between 2 and 10 mm.

The present invention will be more fully understood from the following detailed description of applications thereof, taken together with the drawings, in which:

### BRIEF DESCRIPTION OF THE DRAWINGS

FIG. 1 is a schematic illustration of an anterior view of a healthy native knee joint;

FIG. 2 is a schematic illustration of apparatus for treatment of a native joint of a subject, in accordance with some applications of the present invention;

FIG. 3 is a side view of apparatus for treatment of a native joint of a subject implanted into a native bone of a subject, in accordance with some applications of the present invention;

FIG. 4 is a schematic illustration of apparatus for treatment of a native joint of a subject implanted on either side of the joint, in accordance with some applications of the present invention;

FIG. 5A is a schematic illustration of apparatus for treatment of a native joint of a subject, in accordance with some applications of the present invention;

FIG. 5B is a schematic illustration of a kit comprising apparatus for treatment of a native joint of a subject, in accordance with some applications of the present invention; and

FIG. 6 is a schematic illustration of apparatus for treatment of a native joint of a subject, in accordance with some applications of the present invention.

### DETAILED DESCRIPTION OF EMBODIMENTS

For some applications, a method and apparatus for treatment of a native joint is provided. In this context, in the specification and in the claims, “native joint” means a natural joint rather than a prosthetic joint (i.e., a joint replacement). It is noted that the apparatus and methods described herein are described with reference to a native joint, not a prosthetic joint.

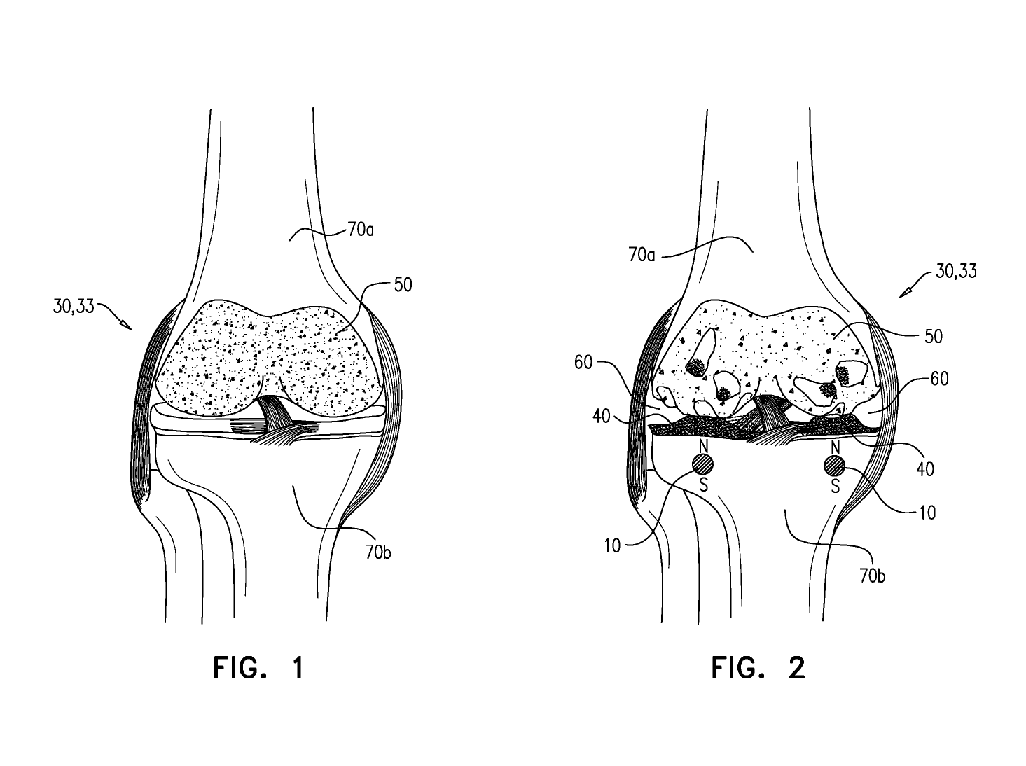

Reference is first made to FIG. 1, which is a schematic illustration of an anterior view of a healthy native joint 30, shown in FIG. 1, as a native knee joint 33. Native joint 30 is shown as knee joint 33 by way of illustration and not limitation. It is noted that the scope of the present invention includes any other synovial joint, e.g., elbows, shoulders and hips. FIG. 1 shows knee joint 33 having cartilage 50 in a healthy state. When cartilage 50 is healthy as shown in FIG. 1, it typically contributes to low-friction motion of articulating bones 70a and 70b in native knee joint 33.

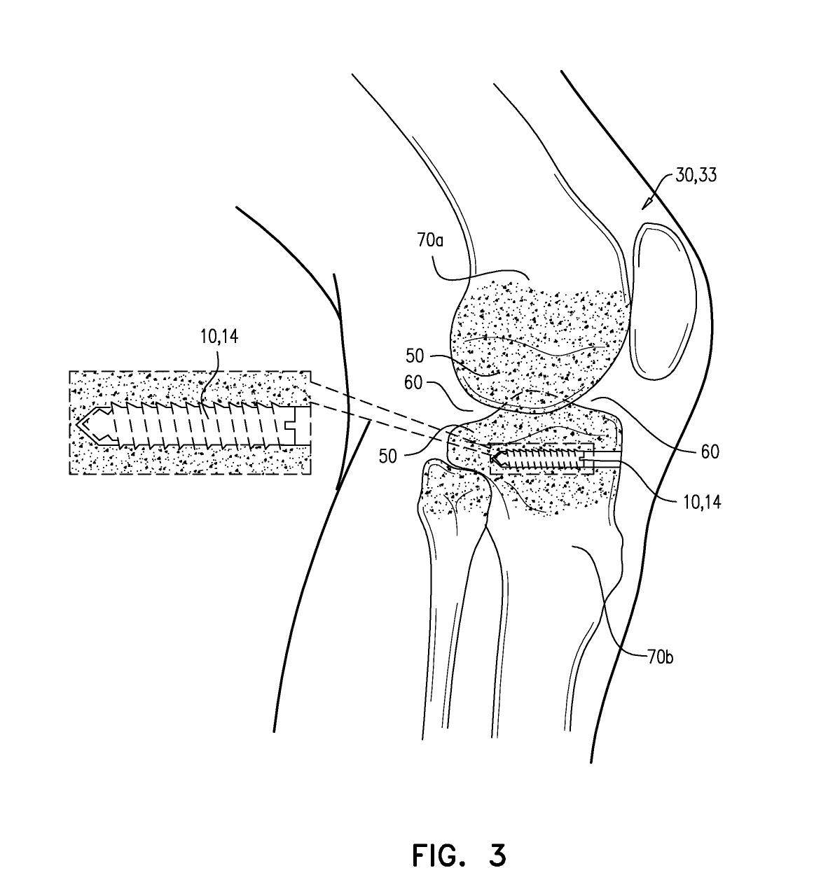

Reference is now made to FIG. 2, which is a schematic illustration of native joint 30, in this case, native knee joint 33, affected by a degenerative joint disease, e.g., osteoarthritis. FIG. 2 shows cartilage 50 in a degenerated state. Typically, erosion of cartilage 50 leads to friction between bones 70a and 70b, resulting in pain and restriction of motion. In accordance with some applications of the present invention, at least one magnet 10 is implanted within a bone (for example, within bone 70a and/or bone 70b) that is adjacent to native knee joint 33, and a ferrofluid 40 is placed in a joint cavity 60 of native knee joint 33. For some applications, injection of ferrofluid 40 is performed during the same procedure as the implantation of magnet 10. Alternatively, ferrofluid 40 may be injected in a separate procedure, subsequently to implantation of magnet 10. Ferrofluid 40 typically acts as a bearing which reduces friction between articulating bones 70a and 70b in native knee joint 33. Magnet 10 typically stabilizes ferrofluid 40, such that at least a portion of ferrofluid 40 is maintained in proximity to magnet 10. Additionally or alternatively, magnet 10 maintains ferrofluid 40 within joint cavity 60 and inhibits leakage of ferrofluid 40 out of joint cavity 60 (and out of the joint capsule).

By reducing friction between articulating bones in native joint 30, ferrofluid 40 typically slows progression of the degenerative joint disease and provides pain relief.

As shown in FIG. 2, in accordance with some applications of the present invention, magnet 10 is implanted within bone 70b in a location that is adjacent to native knee joint 33, typically at a site that is within 3 cm from joint cavity 60 (e.g., 0.1-1 cm from joint cavity 60). As described hereinabove, magnet 10 typically facilitates holding ferrofluid 40 in place within the joint cavity and reduces displacement of ferrofluid 40 within the joint cavity. Typically, placement of ferrofluid 40 in the joint cavity is done by injection. Stabilizing of ferrofluid 40 with magnet 10 typically prolongs the effect of the injection and provides enhanced symptomatic relief to the subject. Additionally, the need for further injections or other treatment options such as joint replacement surgery may be delayed or avoided.

Typically, as shown in FIG. 2, ferrofluid 40 is placed in the joint cavity such that it is not in fluid communication with magnet 10. Optionally, a base liquid of the ferrofluid is hyaluronic acid. (It is known to inject hyaluronic acid into joints to treat degenerative joint disease.) Typically, a volume of 1-10 cc of ferrofluid is placed in the joint cavity.

For some applications, more than one magnet 10, e.g., two magnets 10, are implanted in bone 70b (i.e., in the same bone of the subject), as shown in FIG. 2. Typically, first and second magnets 10 are implanted near each articulating surface of bone 70b. Typically, each one of magnets 10 is implanted within bone 70b adjacent to native knee joint 33, at a site that is within 3 cm from joint cavity 60. The one or more magnets 10 are typically surgically implanted from the anterior side of the bone 70b. Alternatively, one or more magnets 10 are implanted from a lateral or medial side of bone 70b (and/or bone 70a).

For some applications, pre-operative imaging is used to determine the location for implantation of the one or more magnets 10.

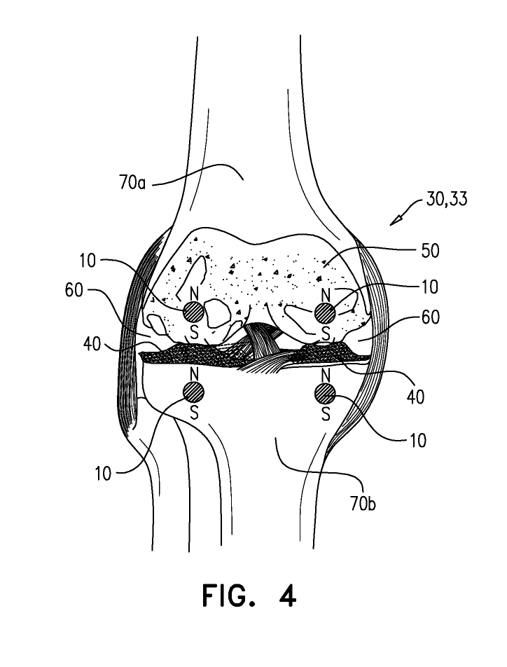

Typically, magnet 10 has a diameter that is between 2 and mm. For some applications, magnet 10 comprises a diametrically-magnetized bone screw, as described hereinbelow with reference to FIG. 3. For other applications, magnet 10 is shaped to define a cylindrical diametrically-magnetized magnet.

For some applications, magnet 10 is coated with a biocompatible coating, e.g., a polymer.

Reference is now made to FIG. 3. For some applications, magnet 10 comprises a diametrically-magnetized bone screw 14 that is typically implanted into bone 70b (and/or bone 70a) by means of screwing bone screw 14 into the bone. Typically, bone screw magnet 14 is implanted within 3 cm, e.g., 0.1-1 cm, from joint cavity 60. Bone screw magnet 14 is typically surgically implanted from the anterior side of the bone 70b by screwing magnet 14 into the bone, or by drilling a hole in the bone and pushing bone screw magnet 14 into the bone. Placement of bone screw magnet 14 into the anterior side of bone 70b is illustrated in side view of joint 33, shown in FIG. 3. Alternatively, bone screw magnet 14 is implanted from a lateral or medial side of bone 70b (and/or bone 70a).

Typically, bone screw magnet 14 has a diameter that is between 2 and 10 mm. For some applications, bone screw magnet 14 is coated with a biocompatible coating, e.g., a polymer.

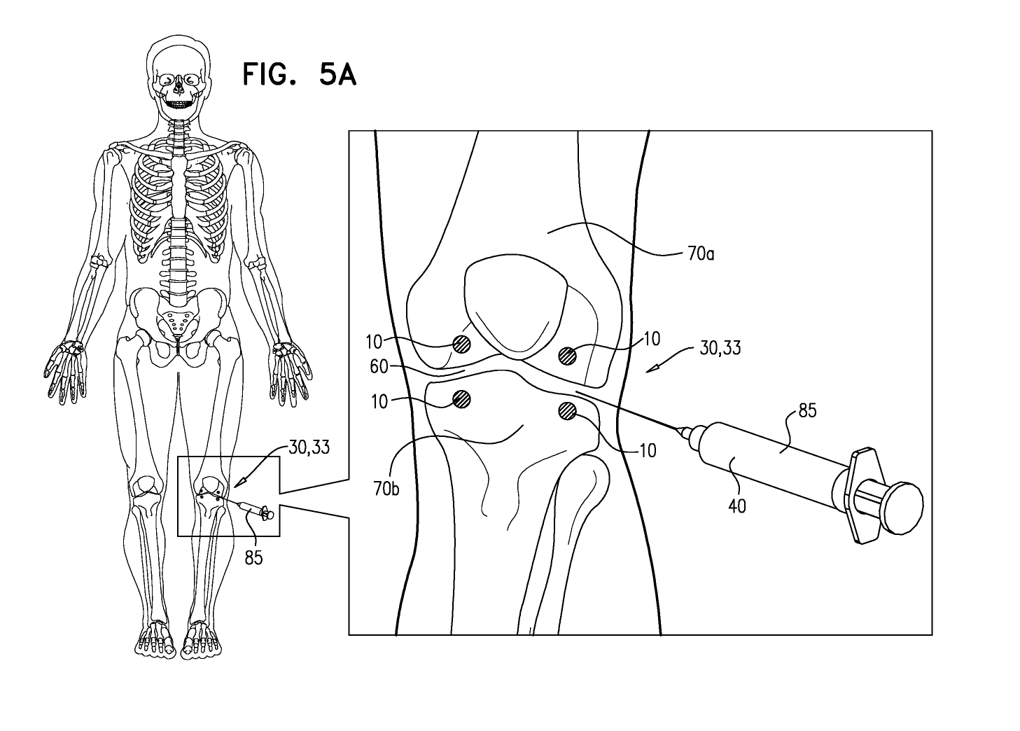

Reference is made to FIG. 4, which is a schematic illustration of a plurality of magnets 10 implanted in separate bones 70a and 70b on either side of native knee joint 33 in order to increase and/or focus the magnetic field. FIG. 4 shows two magnets 10 in each bone by way of example. It is noted that, for some applications, one magnet 10 or alternatively, more than two magnets 10 may be implanted in each one of bones 70a and 70b.

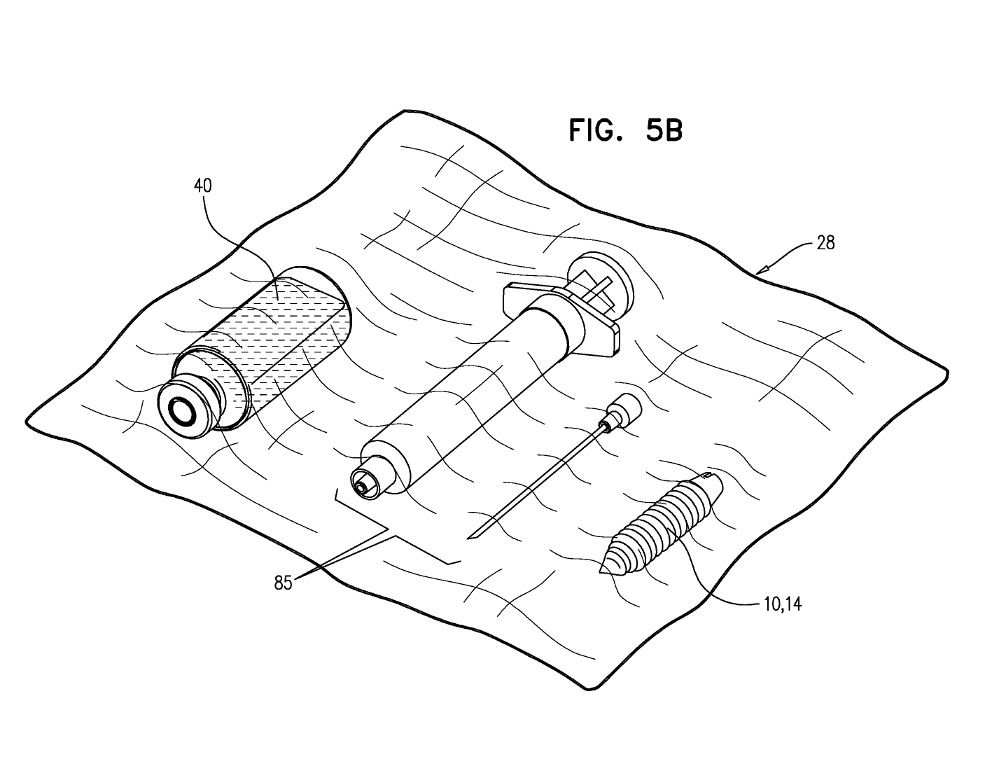

Reference is now made to FIGS. 5A-B. FIG. 5A is a schematic illustration of an injection of a ferrofluid into cavity 60 of native knee joint 33, in accordance with some applications of the present invention. Typically, the ferrofluid is placed into joint cavity 60 by injecting the ferrofluid subsequently to implantation of magnets 10 in the bone. For some applications, syringe 85 is pre-loaded with the ferrofluid. FIG. 5A shows two magnets 10 in each bone by way of example. It is noted that, for some applications, one magnet 10 or alternatively, more than two magnets 10 may be implanted in each one of bones 70a and 70b. Typically, magnet 10 comprises a diametrically-magnetized bone screw 14 as shown in FIG. 3. For some applications, magnet 10 (e.g. bone screw magnet 14), and ferrofluid 40 (optionally within syringe 85) are sold in a kit 28 (FIG. 5B).

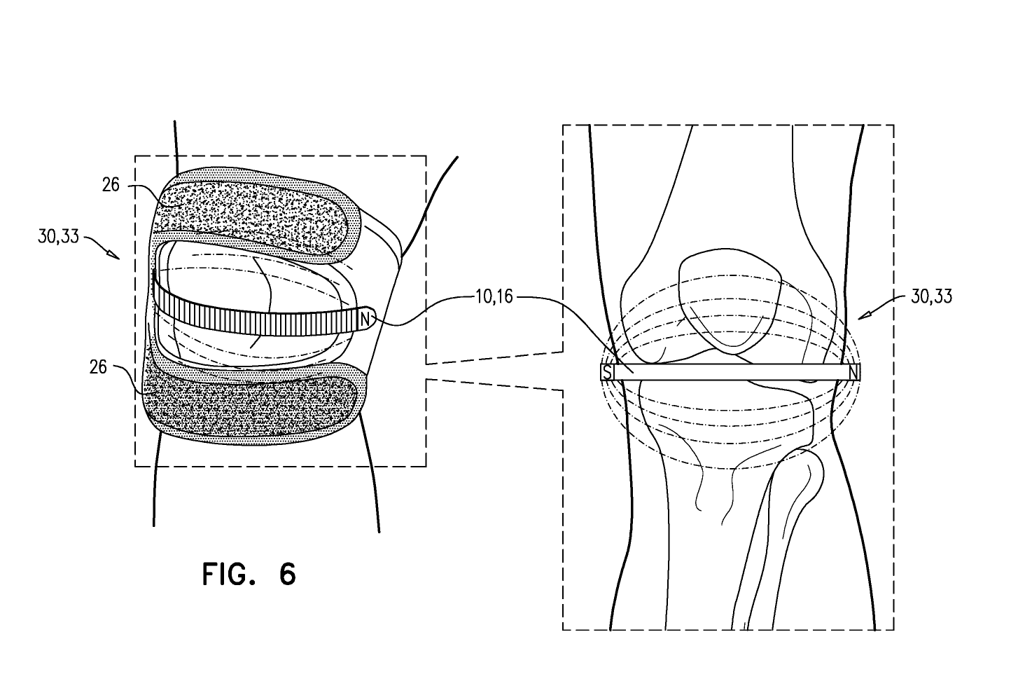

Reference is now made to FIG. 6. FIG. 6 is a schematic illustration of apparatus for treatment of native joint 30 of a subject, in accordance with some applications of the present invention.

For some applications, magnet 10 is coupled to a flexible cuff 26 which is sized and shaped to be worn at least in part around native joint 30, e.g., native knee joint 33. Cuff 26 typically comprises a joint brace, e.g., a knee brace, which extends above and below the joint when worn by the subject. As shown in FIG. 6, for some applications, magnet 10 comprises a curved elongated (e.g., a horse-shoe) magnet 16. Typically magnet 16 is rigid. Alternatively, magnet 16 is flexible. Magnet 16 is coupled to flexible cuff 26 such that when flexible cuff 26 is worn by the subject, magnet 16 is curved around knee joint 33 and is positioned within 8 cm (e.g., within 6 cm) from the joint cavity of native knee joint 33. (It is noted that native joint 30 is shown in FIG. 6 as knee joint 33 by way of illustration and not limitation, and that cuff 26 may be worn around any other synovial joint).

Ferrofluid 40 is typically placed in the joint cavity of the native joint, as described hereinabove with reference to FIGS. 2-5. Ferrofluid 40 is maintained in place within the joint cavity by magnet 16 which is placed externally to native knee joint 33 by being coupled to cuff 26. A magnetic field from magnet 16 typically goes through joint 33, maintaining the ferrofluid at the desired location in the joint cavity. Use of cuff 26 typically avoids implantation of magnet 10 in the bone of the subject by a surgical procedure.

For some applications, more than one magnet 16 is coupled to cuff 26. For example, one magnet 16 may be coupled to, e.g., embedded in, an upper portion of cuff 26 which is placed above native knee joint 33, and a second magnet 16 may be coupled to, e.g., embedded in, a lower portion of cuff 26 which is placed below native knee joint 33. Alternatively or additionally, one magnet 16 is coupled to, e.g., embedded in, a lateral portion of cuff 26 which is placed lateral to native knee joint 33, and a second magnet 16 is coupled to, e.g., embedded in, a medial portion of cuff 26 which is placed medial to native knee joint 33.

Reference is still made to FIG. 6. For some applications, a plurality of magnets are coupled to cuff 26. For example, magnet 16 shown in FIG. 6 may comprise a series of small magnets, thereby contributing to the flexibility of magnet 16. For some applications, the series of small magnets is disposed in a sheath (not shown). Typically, the series of small magnets is arranged such that there is no gap or only a small gap between each magnet. Typically, the gap between each small magnet is less than 5 mm, e.g., less than 1 mm.

For some applications, magnets are coupled to either side of cuff 26 (e.g., where the “N” and “S” are shown in FIG. 6).

Reference is made to FIGS. 2-6. For some applications, magnets 10, 14 and 16 comprise neodymium magnets, by way of example and not limitation. Alternatively, magnets 10, 14 and comprise samarium-cobalt magnets, by way of example and not limitation.

For some applications, methods and apparatus in accordance with some applications of the present invention are used to treat a degenerated disc in a spine of a subject. For such applications, at least one magnet is implanted into a bone adjacent to the disc. Typically, the nucleus of the disc is removed, while keeping the annulus of the disc, and ferrofluid is injected into the disc and is maintained in the disc by the at least one magnet.

It is further noted that methods and apparatus in accordance with some applications of the present invention are used to treat another joint, e.g., a hip, and/or a shoulder, and/or a finger, and/or a toe of a subject.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
a flexible cuff shaped and sized to be worn, at least in part, around a native joint of a subject;
a magnet coupled to the flexible cuff such that when the flexible cuff is worn by the subject, the magnet is positioned within 8 cm from a joint cavity of the native joint; and
a ferrofluid for placing in the joint cavity.

2. The apparatus according to claim 1, wherein the magnet comprises a flexible magnet.

3. The apparatus according to claim 1, wherein the magnet comprises a rigid magnet.

4. The apparatus according to claim 1, wherein the magnet is configured to be positioned within 6 cm from a joint cavity of the native joint.

5. A method for treating a native joint of a subject, comprising:
placing a flexible cuff that includes a magnet around a native joint of a subject such that the magnet is positioned within 8 cm from a joint cavity of the native joint; and
placing a ferrofluid in the joint cavity of the native joint.

6. The method according to claim 5, wherein placing the ferrofluid in the joint cavity comprises placing 1-10 cc of the ferrofluid in the joint cavity.

7. The method according to claim 5, wherein placing the flexible cuff comprises placing the flexible cuff such that the magnet is positioned within 6 cm from the joint cavity.

8. A method for treating a native joint of a subject, comprising:
implanting a magnet within a bone of the subject that is adjacent to the native joint, at a site that is within 3 cm from a joint cavity of the native joint; and
placing a ferrofluid in the joint cavity of the native joint.

9. The method according to claim 8, wherein placing the ferrofluid in the joint cavity does not comprise placing the ferrofluid in fluid communication with the magnet.

10. The method according to claim 8, wherein the native joint is a native knee joint of the subject, and wherein placing the ferrofluid in the joint cavity of the native joint comprises placing the ferrofluid in a joint cavity of the native knee joint.

11. The method according to claim 8, wherein implanting the magnet comprises screwing the magnet into the bone.

12. The method according to claim 8, wherein implanting the magnet comprises implanting a diametrically-magnetized magnet.

13. The method according to claim 8, wherein placing the ferrofluid in the joint cavity comprises placing 1-10 cc of the ferrofluid in the joint cavity.

14. The method according to claim 8, wherein implanting the magnet within the bone comprises implanting the magnet 0.1-1 cm from the joint cavity.

15. The method according to claim 8, wherein the magnet is a first magnet, wherein implanting the magnet within the bone comprises implanting the first magnet, and the method further comprises implanting a second magnet within a bone of the subject that is adjacent to the native joint, at a site that is within 3 cm from the joint cavity.

16. The method according to claim 15, wherein implanting the first magnet and implanting the second magnet comprises implanting the first and second magnets in the same bone of the subject.

17. The method according to claim 15, wherein implanting the first magnet and implanting the second magnet comprises implanting the first and second magnets in separate bones of the subject.

18. The method according to claim 8, wherein implanting the magnet comprises implanting a magnet having a diameter that is between 2 and 10 mm.

19.-21. (canceled)
